(19) United States
(12) Patent Application Publication
(10) Pub. No.: US 2024/0065713 A1
(43) Pub. Date: Feb. 29, 2024

(54) SYSTEMS AND METHODS OF REMOVING CHALLENGING CLOTS AND RESTORING PERFUSION TO A VESSEL

(71) Applicant: Neuravi Limited, Galway (IE)

(72) Inventors: Malgorzata Alicja KOKOSZKA, Galway (IE); David VALE, Barna (IE); Brendan CASEY, Galway (IE); Michael GILVARRY, Headford (IE); Jacqueline O'GORMAN, Clare (IE); Daniel KING, Galway (IE); Kevin McARDLE, Loughrea (IE); Brian FAHY, Galway (IE)

(73) Assignee: Neuravi Limited, Galway (IE)

(21) Appl. No.: 18/237,048

(22) Filed: Aug. 23, 2023

Related U.S. Application Data
(60) Provisional application No. 63/400,240, filed on Aug. 23, 2022.

(51) Int. Cl. A61B 17/221

(52) U.S. Cl. CPC A61B 17/221; A61F 2002/9528

(57) ABSTRACT

A method and/or use of restoring blood flow in neurovasculature by removing thrombus in a plurality of human patients experiencing ischemic stroke using a revascularization device as either a first-line device or a second-line device.

### CROSS-REFERENCE TO RELATED APPLICATION

The present application claims the benefit of priority of U.S. Provisional Application No. 63/400,240, filed Aug. 23, 2022, the contents of which are incorporated herein by reference in its entirety as if set forth verbatim.

### FIELD

This disclosure relates to devices and methods of removing acute blockages from blood vessels.

### BACKGROUND

The World Health Organization estimates that 15,000,000 blood clots occur annually. Clots may develop and block vessels locally without being released in the form of an embolus—this mechanism is common in the formation of coronary blockages. Acute obstructions may include blood clots, misplaced devices, migrated devices, large emboli and the like. Thromboembolism occurs when part or all of a thrombus breaks away from the blood vessel wall. This clot is then carried in the direction of blood flow. The large vessels of the brain include the Internal Carotid Artery (ICA), Middle Cerebral Artery (MCA), Vertebral Artery (VA), and the Basilar Artery (BA). Clots can include a range of morphologies and consistencies. Long strands of softer clot material may tend to lodge at bifurcations or trifurcations, resulting in multiple vessels being simultaneously occluded over significant lengths. Older clot material can also be less compressible than softer fresher clots, and under the action of blood pressure it may distend the compliant vessel in which it is lodged. Clots may also vary greatly in length, even in any one given area of the anatomy. For example, clots occluding the middle cerebral artery of an ischemic stroke patient may range from just a few millimeters to several centimeters in length.

Of the 15,000,000 clots that occur annually, one-third of patients die, and another one-third are disabled. Two of the primary factors associated with mortality in these patients are the occlusion location and the time to treatment. Large-vessel occlusions present in 46% of unselected acute stroke patients presenting in academic medical centers, are associated with higher stroke severity. These more proximal vessels feed a large volume of brain tissue, ergo clinicians use the presenting NIHSS (National Institute of Health Stroke Scale) score as an indicator of large-vessel occlusion.

With this, it is understood that an ischemic stroke may result if the clot lodges in the cerebral vasculature. It is estimated that 87% of stroke cases are acute ischemic stroke (AIS). In the United States alone, roughly 700,000 AIS cases occur every year and this number is expected to increase with an ageing population. Occlusion of these large arteries in ischemic stroke is associated with significant disability and mortality. Revascularization of intracranial artery occlusions is the therapeutic goal in stroke therapy. Endovascular mechanical revascularization (thrombectomy) is an increasingly used method for intracranial large vessel recanalization in acute stroke. Currently, a number of mechanical recanalization devices are in clinical use. First generation devices included the Merci Retriever device. Newer devices based on stent-like technology, referred to as “stentrievers” or “stent-retrievers”, are currently displacing these first generation thrombectomy devices for recanalization in acute ischemic stroke.

There are significant challenges associated with designing clot removal devices that can deliver high levels of performance. There are also a number of access challenges that make it difficult to deliver devices. For example, the vasculature in the area in which the clot may be lodged is often fragile and delicate and neurovascular vessels are more fragile than similarly sized vessels in other parts of the body and are in a soft tissue bed. Excessive tensile forces applied on these vessels could result in perforations and hemorrhage. Pulmonary vessels are larger than those of the cerebral vasculature, but are also delicate in nature, particularly those more distal vessels.

Stent-like clot retriever devices are being increasingly used to remove clots from cerebral vessels of acute stroke patients, but such devices are not without disadvantages. A stent-like clot retriever relies on its outward radial force to grip the clot. If the radial force is too low, the device will lose its grip on the clot. If the radial force is too high, the device may damage the vessel wall and may require too much force to withdraw. Such devices that have sufficient radial force to deal with all clot types may therefore cause vessel trauma and serious patient injury, and retrievers that have appropriate radial force to remain atraumatic may not be able to effectively handle all clot types. In this respect, retriever devices may differ in size, shape, and physical properties, such as radial force, as discussed above, ease of deployment, friction, radiopacity and interaction with vessel wall. See, Loh Y, Jahan R, McArthur D. Recanalization rates decrease with increasing thrombectomy attempts. American Journal of Neuroradiology. 2010 May; 31(5):935-9; and Arai D, Ishii A, Chihara H, Ikeda H, Miyamoto S. Histological examination of vascular damage caused by stent retriever thrombectomy devices, J Neurointerv Surg. 2016 October; 8(10):992-5. Some designs have also been based on in-vitro stroke models that incorporate realistic clot analogs derived from animal blood that represent the wide range of human clots retrieved from stroke patients. See, Eugéne F, Gauvrit J-Y, Ferré J-C, Gentric J-C, Besseghir A, Ronziere T, et al. One-year MR angiographic and clinical follow-up after intracranial mechanical thrombectomy using a stent retriever device, Am. J. Neuroradiol. 2015 January; 36(1):126-32 (18), each of which are incorporated by reference herein in their entirety.

Currently, intravenous (IV) lytics are used for patients presenting up to 4.5 hours after symptom onset. Current guidelines recommend administering IV lytics in the 3-4.5-hour window to those patients who meet the ECASS 3 (European Cooperative Acute Stroke Study 3) trial inclusion/exclusion criteria. Since a large percentage of strokes presenting at hospitals are large vessel occlusions, this is an important clinical challenge to address. Additionally, not all patients may be treated with thrombolytic therapy, and so mechanical thrombectomy is a valuable alternative in patients contraindicated to t-PA (tissue plasminogen activator) or where t-PA treatment was not effective.

Though success rates are high when utilizing mechanical thrombectomy, there are still a proportion of patients for which adequate reperfusion cannot be achieved, certainly, in part, due to the clot not being retrieved. In view of these clear performance disadvantages, further reperfusion and patient outcomes advances in AIS treatment are warranted. Further, there is a need to treat challenging situations where the current stent retrievers are unsuccessful during the first few attempts at clot removal. The solution of this disclosure resolves these and other issues of the art.

### SUMMARY

The subject of this disclosure is the use of a clot revascularization device to treat ischemic stroke for restoring perfusion and/or removing a clot and other obstructions from the neurovascular arteries and veins as well as other vascular beds.

In some examples, a method is disclosed to restore blood flow in neurovasculature of a human patient experiencing ischemic stroke, the method including identifying a human patient within a plurality of human patients at risk of comprising a thrombus; passing a revascularization device as a first-line device to a blood vessel of the respective human patent of a plurality of human patients for retrieving a thrombus; and removing the revascularization device to restore perfusion to the blood vessel and achieve approximately 80% revascularization rate after last pass of the revascularization device under a modified treatment in cerebral infarction score of equal to or greater than grade 2b (mTICI>2b) for the plurality of human patients comprising a thrombus within a predetermined time period of natural stroke symptom onset.

In certain examples, the method can further include detecting, prior to the step of passing the revascularization device as the first-line device, an initial revascularization rate of the respective human patient of the plurality of human patients less than a grade 2b (mTICI<2b).

In some examples, achieving approximately 80% revascularization rate for the plurality of human patients under the modified treatment in cerebral infarction score of equal to or greater than grade 2b (mTICI≥2b) can include less than three passes of the revascularization device as the first-line device. In certain other examples, achieving approximately 80% revascularization rate for the plurality of human patients under the modified treatment in cerebral infarction score of equal to or greater than grade 2b (mTICI≥2b) can include an average of approximately 2.4 passes of the revascularization device as the first-line device.

In some examples, the method can further include reducing, by the step of passing the revascularization device as the first-line device, post-procedural clinical complications selected from at least one of vessel dissection, vessel perforation, hematoma, emboli in new territory, infarction in new territory, or hemorrhagic transformation.

In some examples, the method can be performed within at least 11 hours of stroke symptom onset.

In some examples, the thrombus can be positioned in an internal carotid artery, a M1 segment and/or a M2 segment of a middle cerebral artery, a vertebral artery, or a basilar artery of the human patient.

In some examples, the revascularization device can include a collapsed delivery configuration and an expanded deployed configuration. The revascularization device can also include a proximal pinch section having a spiral shape that includes a spiral pitch. The revascularization device also includes a distal section having a barrel shape.

In some examples, the revascularization device can be configured to position the thrombus against a wall of the blood vessel and then pinch the thrombus with the proximal pinch section.

In some examples, in the expanded deployed configuration, the revascularization device can include peaks of the proximal pinch section that can be laterally spaced-apart. In addition, the revascularization device under tension can be configured to pinch the thrombus between the peaks. In some examples, the proximal pinch section can include a plurality of cells defined by struts and crowns connected to corresponding struts and crowns. At least some of the struts or the crowns of the proximal pinch section can be aligned with a wave-like form to enhance embedding of clot. In some examples, the proximal pinch section can include one or more clot gripping features.

An exemplary embodiment provides a method of restoring blood flow in neurovasculature by removing thrombus in a plurality of human patients experiencing ischemic stroke. The method can include passing a revascularization device by, through, or about a thrombus in a blood vessel of each of the plurality of human patients to restore perfusion to the blood vessel. The method can achieve at least approximately 68.8% revascularization rate after an average of less than two passes of the revascularization device as a second-line device for the plurality of human patients under a modified treatment in cerebral infarction score of equal to or greater than a grade of 2b (mTICI≥2b). The thrombus retrieved can be fibrin-rich and include more fibrin than red blood cells.

In some examples, prior to passing the revascularization device by, through, or about the thrombus in the blood vessel of each of the plurality of human patients, the method can include passing a stent retriever device by, through, or about the thrombus in the blood vessel of each of the plurality of human patients. After the step of passing the stent retriever device by, through, or about the thrombus in the blood vessel of each of the plurality of human patients, and prior to passing the revascularization device by, through, or about the thrombus in the blood vessel of each of the plurality of human patients, the method can include detecting a revascularization rate of a respective human patient of the plurality of human patients less than a grade 2b (mTICI<2b).

In some examples, prior to passing the revascularization device by, through, or about the thrombus in the blood vessel of each of the plurality of human patients, the method can further include switching to the revascularization device as the second-line device to restore perfusion to the blood vessel by passing the revascularization device by, through, or about the thrombus to achieve at least approximately 68.8% revascularization rate after an average of approximately 1.7 passes of the revascularization device for the plurality of human patients under the modified treatment in cerebral infarction score of equal to or greater than a grade of 2b (mTICI≥2b).

In some examples, the revascularization device can include a collapsed delivery configuration and an expanded deployed configuration; a proximal pinch section comprising a spiral shape comprising a spiral pitch; and a distal section comprising a barrel shape. The revascularization device can be configured to position the thrombus against a wall of the blood vessel and then pinch the thrombus with the proximal pinch section.

In some examples, the step of passing the revascularization device can include retracting the revascularization device, after being passed by, through or about the thrombus, while pinching the thrombus.

In some examples, the revascularization device can be configured to remove the thrombus or portions thereof that remain after passing the stent retriever device as a first-line device.

In some examples, the thrombus or portion thereof can include more fibrin than red blood cells.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 shows a patient catheterized via femoral access with an example clot revascularization device positioned in a cerebral vessel using the arterial system for its delivery.

FIG. 2 shows a certain anatomy of cerebral arteries above the aortic arch leading to the brain.

FIG. 3 shows an isometric view of an example device of this disclosure.

FIG. 4 is a table summarizing Thrombolysis in Cerebrovascular Infarction (mTICI) inclusive of the 2c rating for the study of this disclosure.

FIG. 5 is a table summarizing Modified Rankin (mRS) Scale for the study of this disclosure.

FIG. 6 is a table summarizing Heidelberg Bleeding Classification for the study of this disclosure.

FIG. 7 is a table summarizing classification intensity or severity of adverse events assessed in the study of this disclosure.

FIG. 8 shows a table summarizing baseline characteristics of subjects in the study of this disclosure.

FIG. 9 is a table summarizing complications immediately post-procedure for subjects for the study of this disclosure.

FIG. 10 is a table summarizing mechanical thrombectomy passes for subjects for the study of this disclosure.

FIG. 11 is a table summarizing reperfusion and clinical outcomes for the study of this disclosure.

FIG. 12 is a table summarizing clot characteristics for a portion of subject of the study of this disclosure.

FIG. 13 is a table summarizing clot location for the study of this disclosure.

FIGS. 14A and 14B provide a comparison of a gross photograph, histology image (x40), and a histology image (x200) of a typical clot (FIG. 14A) and a tough clot (FIG. 14B) retrieved in this study.

FIG. 15 depicts a graphical overview of one method or use of treating ischemic stroke according to this disclosure.

FIG. 16 depicts a graphical overview of one method or use of treating ischemic stroke according to this disclosure.

FIG. 17 depicts a graphical overview of one method or use of treating ischemic stroke according to this disclosure.

FIG. 18 depicts a graphical overview of one method or use of treating ischemic stroke according to this disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology was resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 99%.

As discussed herein, vasculature of a “subject” or “patient” may be vasculature of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include a doctor, surgeon, or any other individual or delivery instrumentation associated with delivery of a clot revascularization device to the vasculature of a subject.

As discussed herein, “thrombus” can be understood as a clot in the circulatory system that remains in a site of the vasculature hindering or otherwise obstructing flow in a blood vessel. The terms “clot”, “thrombus”, “obstruction”, “occlusion”, “blockage”, and/or the like, can be and are often used interchangeably throughout this disclosure.

Delivery of a “revascularization device” is typically accomplished via delivery of one or more catheters into the femoral artery and/or the radial artery, guided into the arteries of the brain, vascular bypass, angioplasty, and/or the like. “Revascularization devices” can include, but not be limited to, one or more stents, stentrievers, clot removal devices, clot revascularization devices, aspiration systems, one or more combinations thereof, and/or the like, each of which are often used interchangeably throughout this disclosure.

As discussed herein, the term “safety”, as it relates to a clot revascularization device, delivery system, or method of treatment refers to a relatively low severity of adverse events, including adverse bleeding events, infusion, or hypersensitivity reactions. Adverse bleeding events can be the primary safety endpoint and include, for example, major bleeding, minor bleeding, and the individual components of the composite endpoint of any bleeding event.

As discussed herein, unless otherwise noted, the term “clinically effective” (used independently or to modify the term “effective”) can mean that it has been proven by a clinical trial wherein the clinical trial has met the approval standards of U.S. Food and Drug Administration, EMEA or a corresponding national regulatory agency. For example, a clinical study may be an adequately sized, randomized, double-blinded controlled study used to clinically prove the effects of the reperfusion device and related systems of this disclosure. Most preferably to clinically prove the effects of the reperfusion device with respect to an ischemic event, for example, to achieve a clinically effective outcome in for the patient suffering the ischemic event (e.g., mRS less than or equal to 2) and/or achieve reperfusion the vessel(s) afflicted by the ischemic event.

As used herein, unless otherwise noted, the term “predetermined time period” as it relates to a procedure of restoring blood flow in neurovasculature of a human patient means the number of seconds, minutes, hours, days, or week from the onset of natural stroke symptoms until the clinical outcome is achieved for a particular human patient. In some embodiments, the predetermined time period can include performing the procedure of restoring blood flow in neurovasculature within approximately 8, 9, 10, 11, and/or 12 hours of stoke symptom onset.

As discussed herein, the term “computed tomography” or CT means one or more scans that make use of computer-processed combinations of many X-ray measurements taken from different angles to produce cross-sectional (tomographic) images (virtual “slices”) of specific areas of a scanned object, allowing the user to see inside the object without cutting. Such CT scans of this disclosure can refer to X-ray CT as well as many other types of CT, such as positron emission tomography (PET) and single-photon emission computed tomography (SPECT). The present disclosure is related to systems, methods and devices restoring perfusion in blood vessels, and in particular occlusions from cerebral vessels.

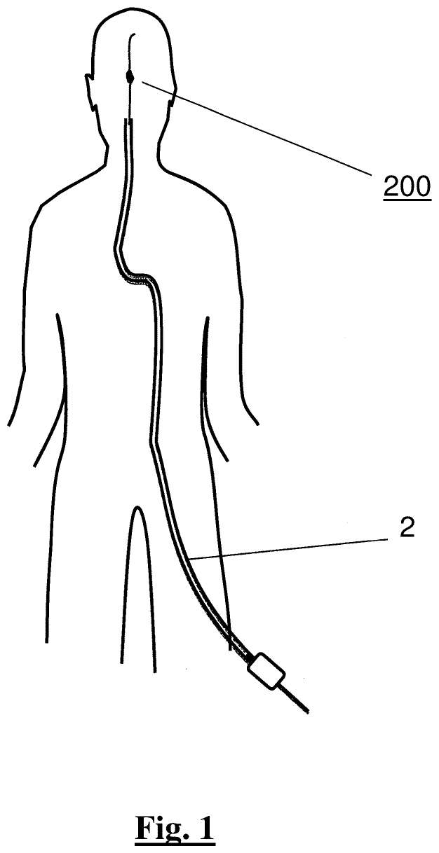

As an example, FIG. 1 depicts a schematic representation of the catheterization of a patient with a clot revascularization device 200, also known as a reperfusion device, via the femoral artery with a catheter 2. Example device 200 is a revascularization device that can restore blood flow in the neurovasculature by removing thrombus in patients experiencing ischemic stroke due to a large vessel neurovascular occlusion (e.g., within 8 hours of symptom onset). Device 200 can be configured for use in the anterior and posterior neurovasculature in vessels of diameter 1.5 mm to 5.0 mm, such as the internal carotid artery, the M1 and M2 segments of the middle cerebral artery, the A1 and A2 segments of the anterior cerebral artery, the basilar, the posterior cerebral and the vertebral arteries. However, it is understood that example device 200 could be used to restore blood flow in less than 8 hours of symptom onset (e.g., 6 hours) or up to 24 hours from symptom onset.

As applicable procedure guidelines change with respect to the use of clot revascularization devices for treatment of ischemic events, it is also conceivable that device 200 could be used more than 24 hours from symptom onset. Device 200 can be understood as including features more clearly described in Appendix 1 of U.S. Provisional Application No. 63/400,240, as incorporated by reference in its entirety from the U.S. Provisional applications from which this application claims priority, which includes U.S. Pat. Nos. 10,292,723; 10,363,054; 10,617,435; 11,253,278; and 11,147,572, each of which are incorporated by reference in their entirety as if set forth verbatim herein. Note that revascularization devices can also be introduced through the wrist artery (radial access) or directly through the carotid artery. While both radial and carotid access avoids the aortic arches, there are other drawbacks. However, all three approaches are considered to be known to ones of skill in this art.

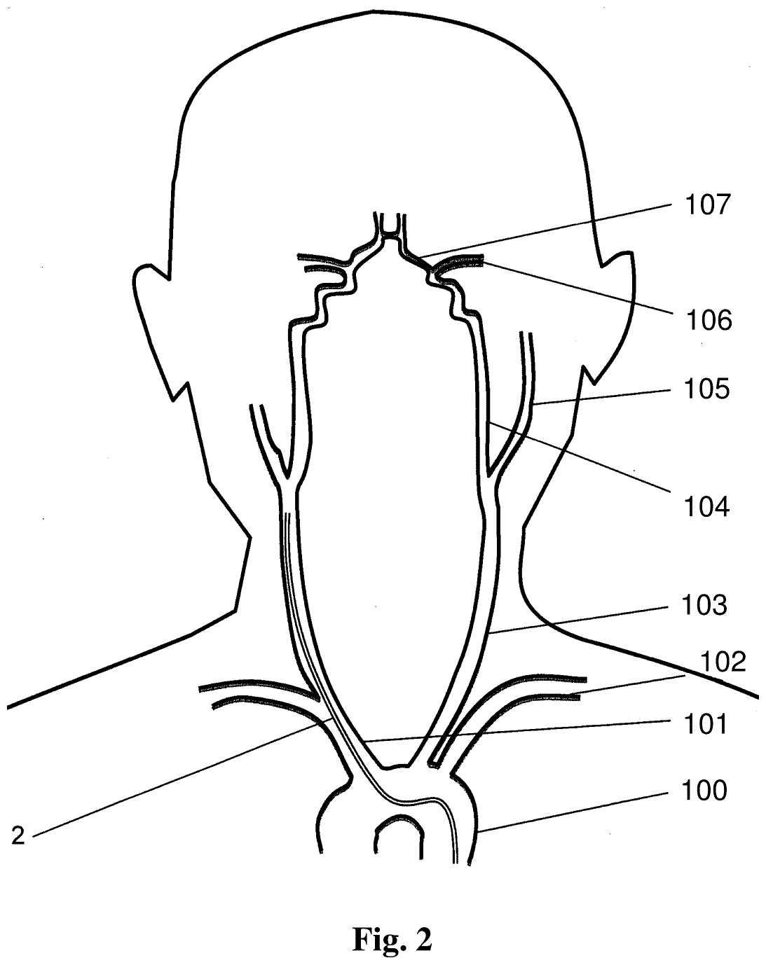

FIG. 2 shows a schematic representation of certain example cerebral vessels. Vessel 100 is the Aorta. Vessel 101 is the brachiocephalic artery. Vessel 102 is the subclavian artery. Vessel 103 is the common carotid artery. Vessel 104 is the internal carotid artery. Vessel 105 is the external carotid artery. Vessel 106 is the middle cerebral artery. Vessel 107 is the anterio-cerebral artery. Catheter 2 from FIG. 1 is shown with its distal end in the common carotid artery. In the more detailed drawings of the invention the details of the access site will not be shown but in general access and delivery is in accordance with FIGS. 1 and 2. Device 200 can be designed for use in the anterior and posterior neurovasculature in vessels such as the internal carotid artery, the M1 and M2 segments of the middle cerebral artery, the vertebral artery, and the basilar arteries. Device 200 can be delivered endovascularly under fluoroscopic guidance in a similar manner to that of other neurovascular clot-retrieval systems.

Once across the site of vessel occlusion, device 200 is deployed to entrap the clot and allow it to be retrieved, hence restoring blood flow. It is understood that device 200 of this disclosure would be used with a delivery system to the site of the clot, including a guide catheter, a microcatheter, and/or a guidewire. It is also contemplated that device 200 of this disclosure could be used in connection with an aspiration system to further facilitate restoring perfusion to the vasculature.

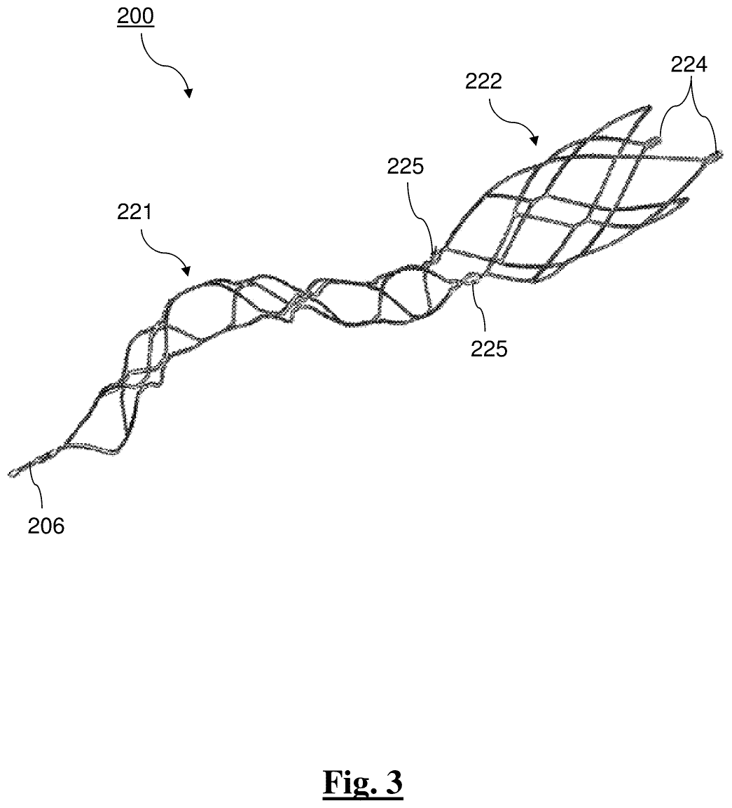

FIG. 3 shows one embodiment of an example clot revascularization device 200 of this disclosure. Device 200 includes a proximal pinch section 221, a distal section 222, distal marker coils 224 and radiopaque markers 225. The proximal pinch section 221 is heat set into a spiral shape, though other shapes of device 200 are contemplated as needed or required. The spiral shape can have a spiral pitch of approximately 14 mm (e.g., within a range of 10 mm to 25 mm). The spiral shape can have a spiral outer diameter of approximately 5 mm (e.g., within a range of 4 mm to 10 mm). The spiral can form a 3600 curve and/or range from 180 to 720°.

A longitudinal center axis of the distal section 222 can be in a barrel shape or otherwise tubular with a lumen and can be offset from a center line of the spiral shape of section 221 to assist in achieving uniform (e.g., low strain) connection between the sections. The distal end of the spiral section is orientated so that it is perpendicular to the proximal face of the barrel section. In this orientation both the struts connecting the spiral section to the barrel section are equal length and have equivalent levels of strain regardless of the cut pattern orientation on the heat forming mandrel. In other iterations the spiral of section 221 can be oriented at an angle to the barrel of section 222.

In some examples, device 200 is configured for removing fibrin rich and/or platelet rich clots. Device 200 can have an expandable structure with a constrained delivery configuration, an expanded clot engaging deployed configuration, and an at least partially constrained clot pinching configuration, whereby at least a portion of the expandable structure is configured to engage the clot in the expanded deployed configuration and to pinch clot on movement from the deployed configuration to the clot pinching configuration. In the clot pinching configuration, device 200 can pinch at least a portion of the clot body as its expandable element is at least partially collapsed from a fully expanded configuration. The expandable element of device 200 can be configured to come into contact with at least a portion of the clot, while maintaining the position of a shaft (e.g., shaft 206) steadfast and effecting pinching substructure of the device so as to pinch at least a portion of the clot and retracting device 200 and the pinched occlusive clot from the patient.

Device 200 can have an elongate shaft 206. Shaft 206 can have a distal end that extends interior of the artery and a proximal end that extends exterior of the artery. Shaft 206 may be a tapered wire shaft, and may be made of stainless steel, MP35N, Nitinol or other material of a suitably high modulus and tensile strength. Shaft 206 can have a coil adjacent to its distal end and is proximal of the outer member and inner tubular member. The coil may be coated with a low friction material or have a polymeric jacket positioned on the outer surface.

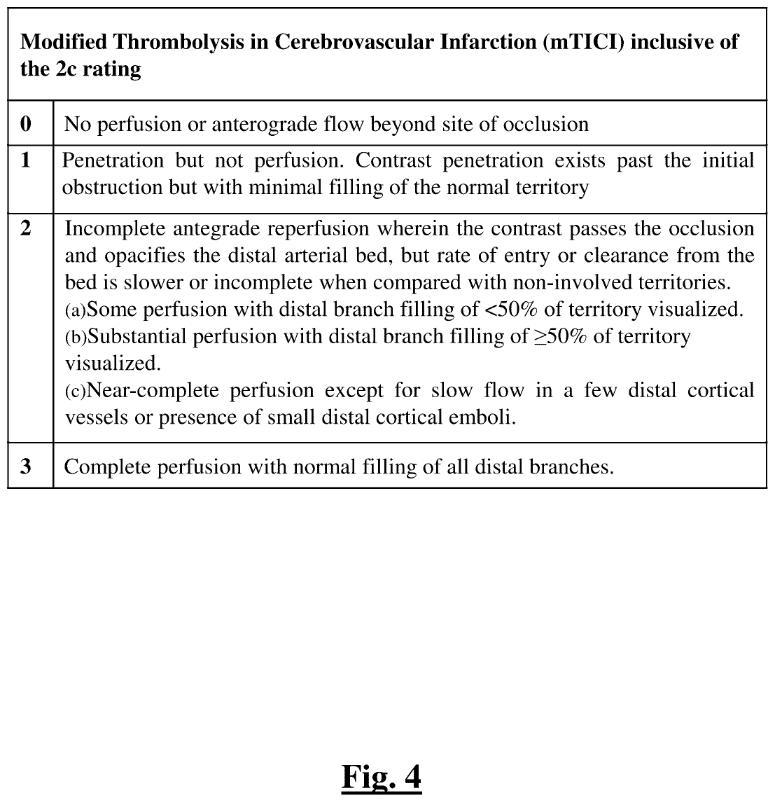

FIG. 4 is a table summarizing Thrombolysis in Cerebrovascular Infarction (mTICI) inclusive of the 2c rating for the study of this disclosure. As discussed herein, “mTICI” means modified thrombolysis in cerebral infarction (TICI) score. An mTICI score of 0 means no perfusion. An mTICI score of 1 means antegrade reperfusion past the initial occlusion but limited distal branch filling with little or slow distal reperfusion. An mTICI score of 2 generally means incomplete antegrade reperfusion wherein the contrast passes the occlusion and opacifies the distal arterial bed but there are residual antegrade perfusion deficits. More particularly, an mTICI score of 2a means antegrade reperfusion of less than half of the occluded target artery previously ischemic territory (e.g., in 1 major division of the MCA and its territory). An mTICI score of 2b means antegrade reperfusion of more than half of the previously occluded target artery ischemic territory (e.g., in 2 major divisions of the MCA and their territories). An mTICI score of 2c means antegrade reperfusion of >90% but less than TICI 3 or near complete reperfusion. An mTICI score of 3 means full perfusion with filling of all distal branches.

It is noted, however, that other measures of cerebral scoring standards, such as expanded TICI (eTICI), other known and/or to-be-developed cerebral scoring standards, provide measures of cerebral scoring and are thus directly and/or indirectly applicable in understanding scope of the presently disclosed solution. eTICI scale is a 7-point compilation of TICI grades that reflects all previously reported thresholds used to define reperfusion after endovascular stroke therapy. For example, eTICI grade 0, just as mTICI, can be equivalent to no reperfusion or 0% filling of the downstream territory. eTICI 1 can indicate thrombus reduction without any reperfusion of distal arteries, including reperfusion of less than half or 1-49%. eTICI of 2b50 can be 50-66% reperfusion. eTICI 2b67 can be 67-89% reperfusion, exceeding TICI but below TICI2C. eTICI 2c can be equivalent to TICI 2C or 90-99% reperfusion. eTICI 3 can be complete or 100% reperfusion, such as TICI 3. It is understood that one of ordinary skill in the art can also correlate between currently known cerebral scoring standards and/or to-be-developed cerebral scoring standards (e.g., from mTICI to eTICI).

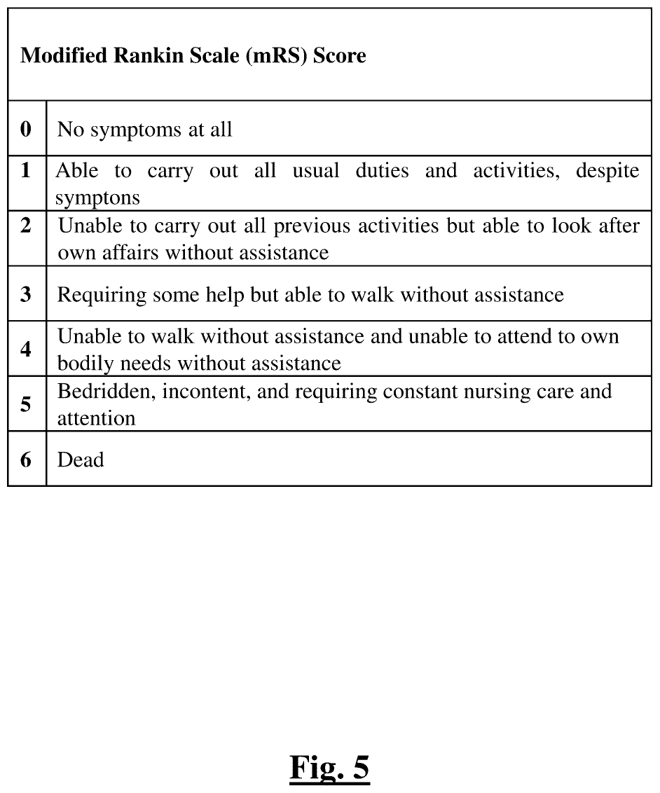

FIG. 5 is a table summarizing Modified Rankin (mRS) Scale for the study of this disclosure. As discussed herein, “mRS” means the modified Rankin Scale (mRS) that is a commonly used scale for measuring the degree of disability or dependence in the daily activities of people who have suffered a stroke or other causes of neurological disability. The mRS scale runs from 0-6, running from perfect health without symptoms to death. An mRS score of 0 is understood as no symptoms being observed. An mRS score of 1 is understood as no significant disability is observed and the patient is able to carry out all usual activities, despite some symptoms. An mRS score of 2 is understood as slight disability and the patient is able to look after their own affairs without assistance, but unable to carry out all previous activities. An mRS score of 3 is understood as moderate disability whereby the patient can require some help but is able to walk unassisted. An mRS score of 4 is understood as moderate severe disability and the patient is unable to attend to their own bodily needs without assistance or walk unassisted. An mRS score of 5 is understood as severe disability and the patient requires constant nursing care and attention, bedridden, incontinent. An mRS score of 6 is understood as the patient being deceased.

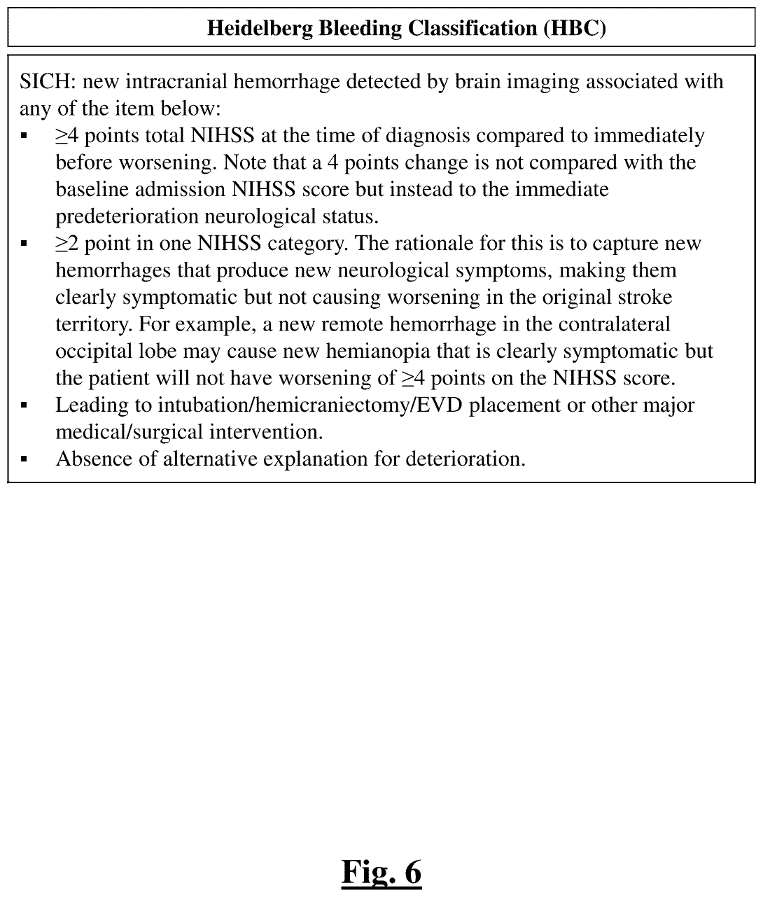

FIG. 6 is a table summarizing Heidelberg Bleeding Classification (“HBC”) for the study of this disclosure. The HBC is a classification of bleeding events after reperfusion therapy and includes both a pure radiological classification scheme and a combined radiological-symptomatic classification scheme. The pure imaging scheme provides an objective comparison among different series, based solely on radioanatomic features of the hemorrhage. Assessment of impact on the clinical course, in contrast, will inevitably be more variable across studies, depending on the intensity of monitoring for early neurological worsening and validity of functional outcome assessments. By conducting routine follow-up brain imaging with either CT or MRI at baseline and within 48 hours after reperfusion treatment, a neurological deterioration greater than or equal to 4 points NIHSS compared to NIHSS at baseline is noted by the HBC as a relevant change in neurological symptoms. The HBC intracranial hemorrhage classification is able to (1) capture and describe all types of intracranial hemorrhages; (2) differentiate between asymptomatic (which may be irrelevant for prognosis) and symptomatic intracranial hemorrhage, “sICH”; (3) help to assess the relatedness between intracranial hemorrhage and therapeutic intervention; (4) be simple to administer and reduce costs; (5) have a high inter-rater reliability; and (6) be easily understandable for clinicians and patients to allow easy communication of risk. As discussed herein, “NIHSS Score” means The National Institutes of Health Stroke Scale, or NIH Stroke Scale (NIHSS) and is a tool used by healthcare providers to objectively quantify the impairment caused by a stroke. The NIHSS is composed of 11 items, each of which scores a specific ability between a 0 and 4. For each item, a score of 0 typically indicates normal function in that specific ability, while a higher score is indicative of some level of impairment. The individual scores from each item are summed in order to calculate a patient's total NIHSS score. The maximum possible score is 42, with the minimum score being a 0.

As discussed herein, symptomatic intracranial hemorrhage, “sICH” is any extravascular blood in the brain or within the cranium associated with clinical deterioration, as defined by an increase of 4 points or more in the score on the NIHSS, or that leads to death and is identified as the predominant cause of the neurologic deterioration. For the purpose of this disclosure, subjects with sICH identified through all posttreatment scans up to the 24-hour time-point (including those performed due to clinical deterioration), were considered in the study discussed herein.

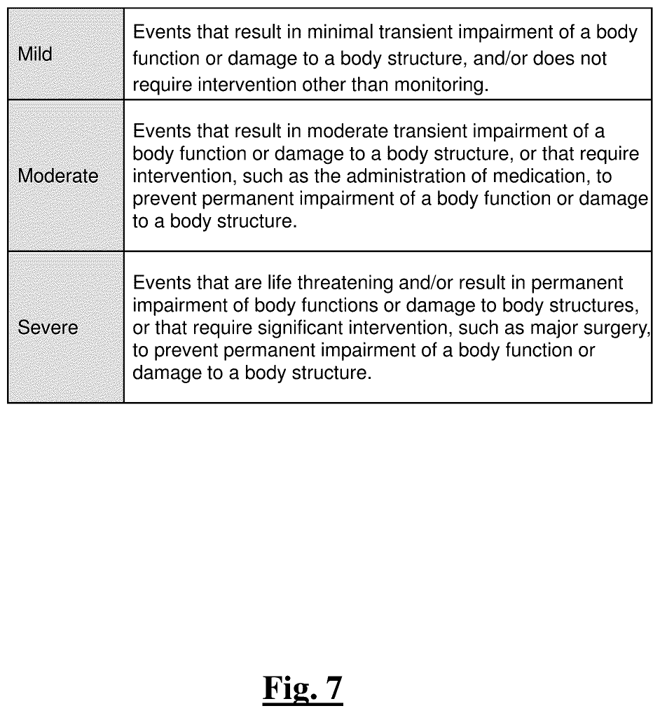

FIG. 7 is a table summarizing classification intensity or severity of adverse events assessed in the study of this disclosure. A mild adverse event includes events that result in minimal transient impairment of a bodily function or damage to a bodily structure. A mild adverse event does not require intervention other than monitoring of the patient. A moderate adverse event includes events that result in moderate transient impairment of a bodily function or damage to a bodily structure. With a moderate adverse event, intervention such as administration of medication may be required to prevent permanent impairment to the patient. A severe adverse event includes events that are life threatening and/or may result in permanent impairment of bodily functions or damage to bodily structures. A serious adverse event requires significant intervention, such as major surgery, to prevent permanent impairment to the patient.

### Study Overview

This disclosure is more clearly understood with a corresponding study discussed more particularly below with respect to treatment of ischemic stroke, which is in Appendix 2 of U.S. Provisional Application No. 63/400,240, which is incorporated by reference in its entirety as if set forth verbatim herein. In the study of this present disclosure, real-world outcomes and clot composition using device 200 for challenging clots were assessed in a retrospective review of patients treated at two high-volume comprehensive stroke centers. In particular, the study assessed consecutive patients with AIS who underwent MT using the device 200 at 2 high-volume centers from December 2019 to May 2021. Use of device 200 was at the interventionalist's discretion at the time of the procedure and could be deployed as first-line treatment for clots deemed challenging to remove or after failed attempts using standard techniques. The study was approved by the local ethics committee. Informed consent was waived owing to the retrospective nature of the study.

It is understood that data and study information is presented herein for purposes of illustration and should not be construed as limiting the scope of the disclosed technology in any way or excluding any alternative or additional embodiments. Data was collected at baseline (prior to thrombectomy), during the procedure, and post-procedure. In general, device 200 is traditionally used in procedures for remove tough clots after prior devices have failed to remove the tough clot. The use of device 200 as a first-line device has not been done before. Therefore, the objective of this study was to assess the efficacy of device 200 in a real-world setting with patients where device 200 was used as a first-line device and/or the first two passes with another stent-retriever did not achieve an mTICI score of 2b or better. The rate of effectiveness achieved was considered as well as clot characteristics (e.g., Composition of clot components, per pass, Red Blood Cells (RBC), White Blood Cells (WBC), platelets, fibrin, and other proteins), as evaluated by the independent Central Lab and clinical outcomes. The study was a retrospective study that evaluated real-world outcomes using device 200 for challenging clots at two high-volume comprehensive stroke centers. In particular, the study evaluated device 200 as either a first-line device (5 subjects out of 37 total subjects, 13.5%) or second-line device after two unsuccessful passes of another stent-retriever (32 subjects out of 37 total subjects), in the treatment of acute ischemic stroke.

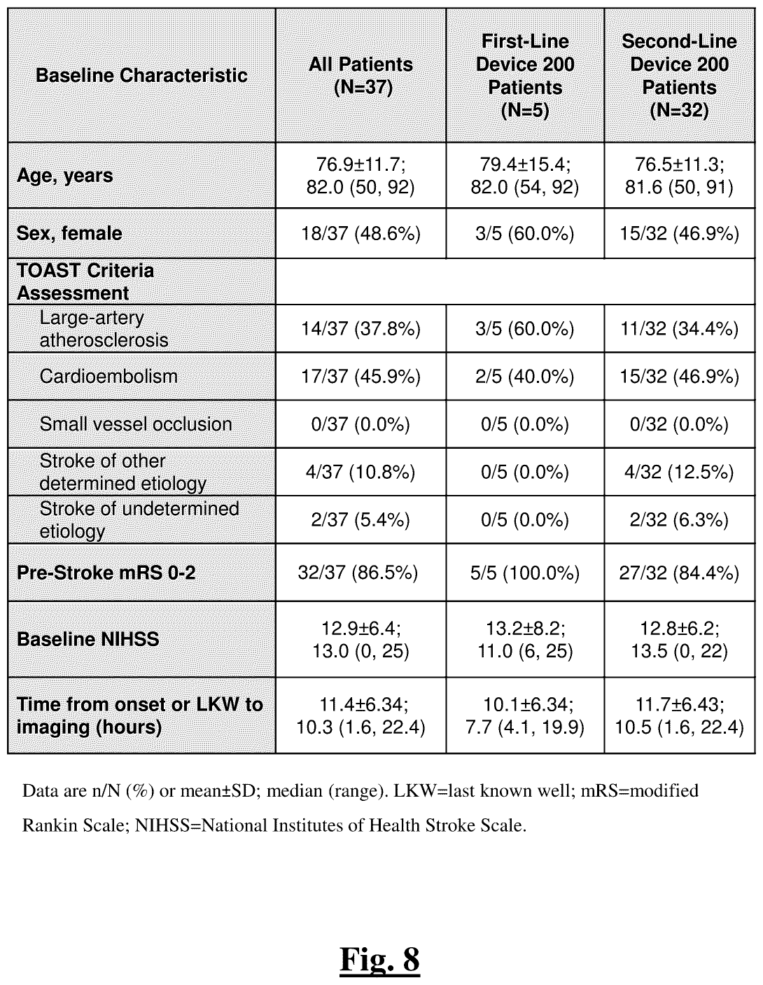

FIG. 8 of this disclosure shows a summary of baseline, demographic information of subjects of the study with device 200 used as either first line or second line for retrieving tough clots. As illustrated, during December 2019 to May 2021, 37 patients underwent mechanical thrombectomy procedures using device 200. Disease state baseline characteristics were classified according to TOAST (Trial of ORG 10172 in acute stroke treatment) criteria, described in more detail in Adams H P, Jr., Bendixen B H, Kappelle L J, et al. Classification of subtype of acute ischemic stroke. Definitions for use in a multicenter clinical trial. TOAST. Trial of Org 10172 in Acute Stroke Treatment. Stroke 1993; 24:35-41. Out of all the patients, the largest TOAST criteria included a subtype of AIS of cardio embolism in 45.9% (17/37), followed by subtype of large-artery atherosclerosis in 37.8% (14/37). A lower number of patients presented a stroke of other determined etiology (10.8%; 4/37) or a stroke of undetermined etiology (5.4%; 2/37). No patients of the present study had a small vessel occlusion subtype.

Using standard interventional techniques, access to the arterial system and using angiography, the location of the occluded vessel was determined. Then, an appropriate guide catheter, sheath or balloon guide catheter was advanced as close to the occlusion site as possible. In some examples, rotating hemostasis valve (RHV) was connected to the proximal end of this catheter and a continuous flush system. With the aid of a suitable guidewire, and using standard catheterization techniques and fluoroscopic guidance, an appropriately sized microcatheter was advanced up to and across the occlusion so that the distal end of the microcatheter was positioned distal to the occlusion. The guidewire was removed, and device 200 was inserted and advanced into the microcatheter. In some examples, immediately prior to introducing a guide catheter, the physician in the study performed an angiogram of the affected intracranial artery. The purpose of the pre-procedure angiogram was to confirm the location of the occlusion; that the subject remained suitable for treatment with mechanical thrombectomy; and that the subject remains a candidate for the study per the eligibility criteria.

In some examples, device 200 continues to be advanced until radiopaque distal markers of device 200 approach the distal region of the microcatheter. Device 200 was positioned in the clot ideally such that the end of the proximal radiopaque coil was aligned with the proximal face of the clot. To fully deploy device 200 within the clot, the microcatheter was retracted until the distal tip of the microcatheter was positioned over the proximal radiopaque coil of device 200.

The primary endpoint was successful revascularization at the end of the procedure, without rescue as determined by an Independent Core Lab, where the successful revascularization is defined as achieving an mTICI score of 2b or greater. Revascularization was measured using modified Thrombolysis in Cerebrovascular Infarction (“mTICI”) inclusive of the 2c rating that is described in FIG. 4. Two-sided exact 95% confidence intervals were conducted around the percentage. Subjects who received any rescue therapy prior to the date of 90-day follow-up, were included in the analysis, but considered as not to have achieved this endpoint. Subjects with missing data on mTICI were excluded from the analysis.

Other endpoints of the study included evaluating successful revascularization (final mTICI≥2c), which was understood as the rate of achieving an mTICI score of 2c or greater at the end of the procedure, as determined by an Independent Core Lab; first study pass (third procedural pass) recanalization (mTICI≥2b), which was understood as the rate of achieving an mTICI score of 2b or greater after the third procedural pass, as determined by an Independent Core Lab with two-sided exact 95% confidence intervals conducted around the percentage; Occurrence of Embolization to a New Territory (ENT), which was understood as the rate of embolization in a previously unaffected territory, following the fifth procedural pass, or the final procedural pass, if earlier; pre-stroke modified Rankin Scale (mRS) scores (as provided in FIG. 5) and National Institutes of Health Stroke Scale (NIHSS) scores; and Symptomatic Intracerebral Hemorrhage (sICH) at 24 hours specified according to the Heidelberg Bleeding Classification (HBC), which was understood as the rate of sICH at 24 hours post-procedure. Symptomatic Intracerebral Hemorrhage (sICH) is defined per the Heidelberg Bleeding Classification, as in FIG. 6.

Key assessments of clot retrieval by device 200 included number of passes (prior to device 200, device 200 only and all devices), substantial reperfusion (modified Thrombolysis in Cerebral Infarction (“mTICI”) 2b or higher prior to device 200, after last device 200 pass and with all devices), rate of any clot material retrieval (“non-empty” passes) for device 200 vs. earlier MT passes, and use of device 200 in the final pass where end-of-procedure substantial reperfusion was achieved. Reasons for using or switching to device 200 were also recorded.

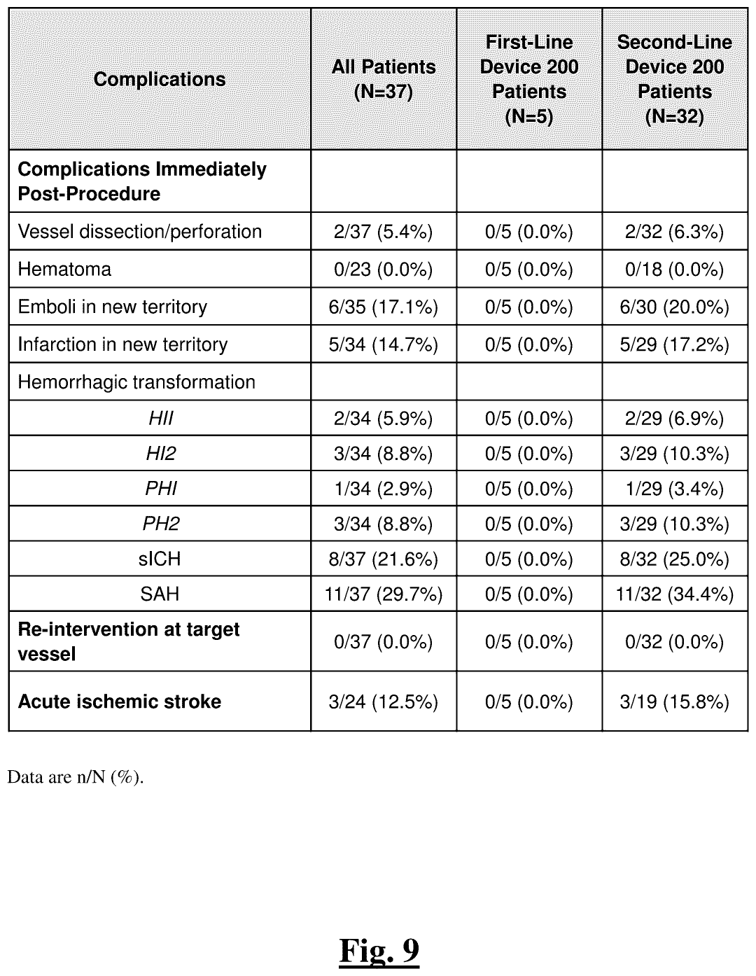

Clinical outcomes included NIHSS change from baseline at 48 hours post-procedure, good (0-2) and fair (0-3) mRS outcomes (discharged to 7 days after the procedure and at final assessment) and all-cause mortality during primary hospitalization. As outlined in FIG. 9, safety outcomes included complications immediately post-procedure, such as vessel dissection/perforation, hematoma, emboli in new territory, infarction in new territory, hemorrhagic classification, intracerebral hemorrhage (ICH), and subarachnoid hemorrhage (SAH). Incidences of re-intervention at the target vessel and AIS were also captured. Of all patients receiving device 200 as first-line treatment, no patients experienced complications immediately post-procedure. When using a comparative device as first-line treatment and device 200 as second-line treatment, 20% (6/30) of patients experienced an embolus in a new territory immediately post-procedure; 17.2% (5/29) of patients experienced infarction in new territory; and 6.3% (2/32) of patients experienced vessel dissection and/or perforation. Complications that were presented when device 200 was used as the second line device indicate that device 200 was not the only device used in those cases and the complications cannot be attributed to device 200 alone or any individual device.

One of the centers had the capacity to collect clot material in 78.3% (18/23) cases. Retrieved clots were collected per pass, immediately fixed in 10% phosphate buffered formalin and shipped to an independent lab for histological analysis including Martius Scarlet Blue staining, as previously described in Duffy S, McCarthy R, Farrell M, et al. Per-Pass Analysis of Thrombus Composition in Patients with Acute Ischemic Stroke Undergoing Mechanical Thrombectomy. Stroke 2019; 50:1156-1163. Upon arrival, gross photos were taken of all clots and the extracted clot area was measured using ImageJ as previously described in Rossi R, Fitzgerald S, Gil S M, et al. Correlation between acute ischaemic stroke clot length before mechanical thrombectomy and extracted clot area: Impact of thrombus size on number of passes for clot removal and final recanalization. Eur. Stroke J. 2021; 6(3):254-261. Following standard tissue processing and paraffin embedding protocols, clots were cut into 3 μm sections and stained. Orbit image analysis was used to quantify the histological composition (red blood cells, white blood cells, fibrin, platelets/other).

The study collected imaging data as assessed by Imaging Core Lab, including Baseline—CT/MR imaging (e.g., Infarct volume, Clot location, Clot length, Clot radiodensity on CT/Susceptibility Vessel Sign (SVS) on MRI), procedural angiography (e.g., mTICI score for every pass, clot location for every pass (proximal face of clot), emboli to new territories), post procedure as to CT/MR imaging (e.g., intracranial hemorrhage). Hemorrhages were classified according to the following categories HI 1—Scattered small petechiae, no mass effect; HI 2—Confluent petechiae, no mass effect; PH1—Hematoma within infarcted tissue, occupying <30%, no substantive mass effect; PH2—Hematoma occupying 30% or more of the infarcted tissue, with obvious mass effect; RIH—Parenchymal hematoma remote from infarcted brain tissue; IVH—Intraventricular Hemorrhage; SAH—Subarachnoid Hemorrhage; and SDH—Sub Dural Hemorrhage. Reperfusion as a primary endpoint was not assessed by the Core Lab.

Prior to clot retrieval, the microcatheter was re-advanced to the clot while holding device 200 push wire static until a predetermined resistance was met. If the operator felt significant resistance, she did not continue to advance. Device 200 was withdrawn with microcatheter slowly and carefully as a single unit to the guide catheter while aspirating through the guide and maintaining microcatheter and device 200 position relative to each other during the withdrawal step. In some examples, vigorous aspiration was applied by syringe and device 200 withdrawn with microcatheter into the guide catheter and continue to aspirate until device 200 reached the RHV on the guide. The operator could then disconnect the RHV from the guide and remove device 200, microcatheter and RHV together from the guide. Device 200 was used for up to three retrieval attempts. If an additional pass was to be made with device 200, then any captured thrombus was removed from device 200, and device 200 was cleaned in heparinized saline, rubbing gently from proximal to distal to remove any residual thrombus material.

### Patient Selection

Subjects presenting with AIS were evaluated and treated by the physician according to institutional practice. Endovascular treatment with device 200, and the other stent-retrievers, was performed per hospital standard technique and in accordance with the applicable devices' IFU. As outlined in FIG. 8, across both groups, the patients ranged in age from 50 to 92 years (mean age 76.9±11.7; median 82 years), and 48.6% (18/37) were female. The etiology for the majority of patients was either cardio embolism (17/37 [45.9%]) or large-artery atherosclerosis (14/37 [37.8%]). The average NIHSS score at presentation was 12.9±6.4 with an average time since stroke onset or last known well of 11.4±6.34 hours.

### Clot Retrieval by Device 200

The most common reason for using device 200 (32/37 cases) involved failure of up to 4 devices in up to 6 thrombectomy passes. Reasons for using device 200 as first-line treatment included suspected calcified lesion (3/37), known M1 stenosis (1/37) and intracranial internal carotid artery (ICA) stenosis (1/37).

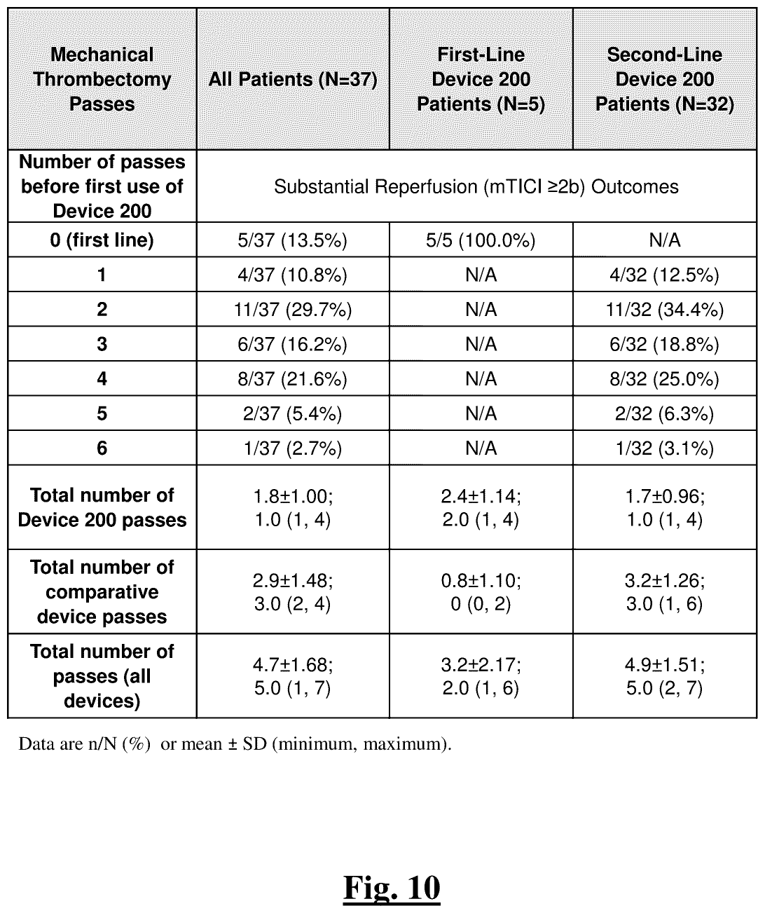

As outlined in FIG. 10, prior to switching to device 200, an average of 1.2±0.59 devices were used in a mean 2.9±1.3 number of passes and 17/32 (53.1%) patients had undergone three or more passes with an outcome of 0/32 (0%) mTICI≥2b before the first use of device 200. Substantial reperfusion (mTICI≥2b) was achieved in 29/37 patients (78.4%) with a mean 1.8±1.0 device 200 passes (mean 4.7±1.7 passes with all devices) and device 200 was the final device used in 79.3% (23/29) of those cases. Based on 23 patients in whom this data was captured, the rate of any clot material retrieval with each device 200 pass was 88.1% compared to 40.0% in prior thrombectomy maneuvers.

As shown in FIG. 10, when device 200 was used as the first-line therapy, all patients (5/5) reached substantial reperfusion (mTICI≥2b) outcomes with an average number of 2.4±1.14 passes in a mean of 2 passes. When device 200 was used as a second-line therapy, a comparative device, or stent retriever, was passed an average number of 3.2±1.26, with a mean of 3 passes, before switching to device 200. In particular, 12.5% (4/32) of patients underwent at least 1 pass of a comparative device before switching to device 200; 34.4% (11/32) of patients underwent 2 passes of a comparative device before switching to device 200; 18.8% (6/32) of patients underwent 3 passes of a comparative device before switching to device 200; 25% (8/32) of patients underwent 4 passes of a comparative device before switching to device 200; 6.3% (2/32) of patients underwent 5 passes of a comparative device before switching to device 200; and 3.1% (1/32) of patients underwent 6 passes of a comparative device before switching to device 200.

### Clinical Outcomes

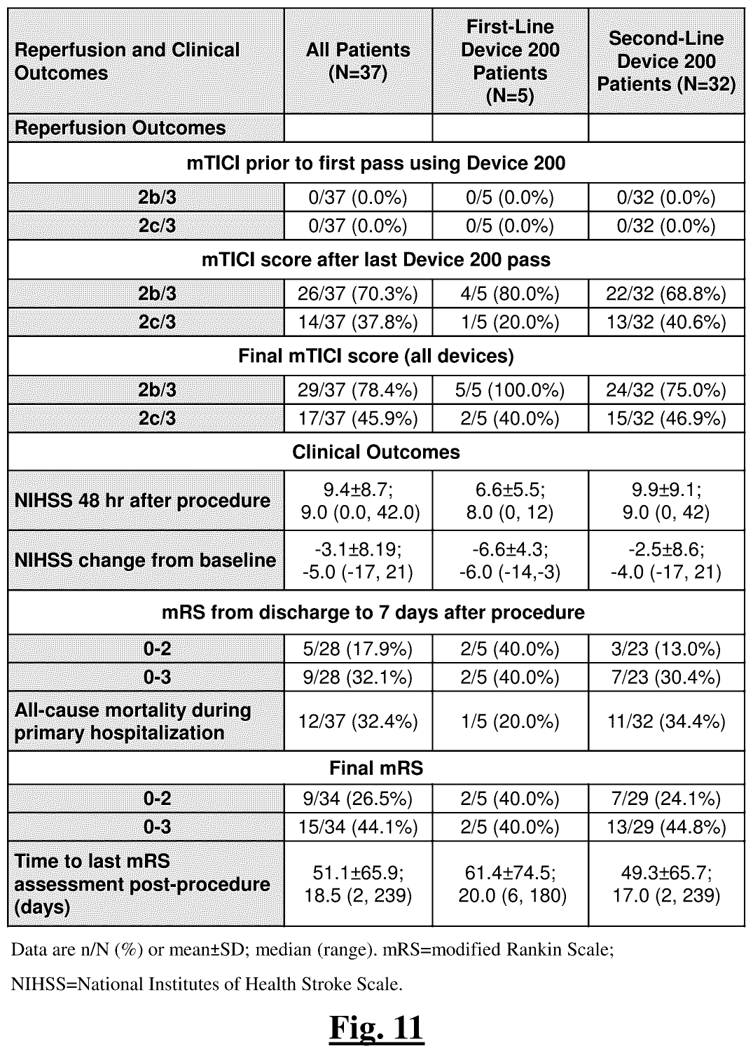

Reperfusion and clinical outcomes are summarized in FIG. 11. The mean NIHSS score 48 hours post-procedure was 9.4±8.7, with a mean improvement of 3.1±8.19 points compared to baseline. A total of 5/28 (17.9%) patients had an mRS score of 0-2 from discharge to day 7 post-procedure, and 9/34 (26.5%) patients had an mRS score of 0-2 at final assessment. All-cause mortality during primary hospitalization was 32.4% (12/37). As shown in FIG. 9, none of the patients in whom device 200 was used as first-line treatment experienced clinically significant complications. In addition, when device 200 was used as first-line treatment, the mean NIHSS score 48 hours post-procedure was 6.6±5.5, with a mean improvement of 6.6±4.3 points compared to baseline. A total of 2/5 (40%) patients had an mRS score of 0-2 from discharge to day 7 post-procedure, and 2/5 (40%) patients had an mRS score of 0-2 at final assessment. All-cause mortality during primary hospitalization was 20% (1/5).

Prior to passing device 200, all 37 patients had an mTICI score lower than 2b, but the mTICI score equal to or greater than 2b after last pass of device 200 reached 70.3% (26/37) of patients, 37.8% (14/37) of which had an mTICI score equal to or greater than 2c.

### Clot Composition Analysis

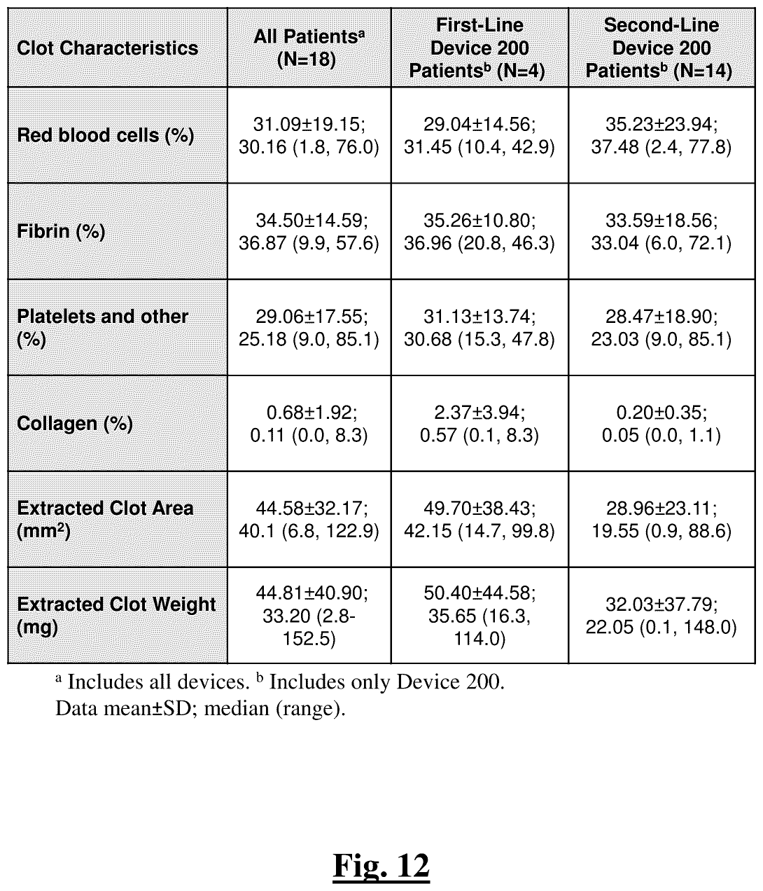

Clot specimens from 18/37 (48.6%) cases that underwent composition analysis are summarized in FIG. 12. In individual fragments for all patients, fibrin levels (mean of 34.5±14.59%), were slightly higher than red blood cell levels (31.09±19.15%) and platelet/other component levels (29.06±17.55%). When clot specimen underwent composition analysis, fibrin and platelets represented 31.4±13.7% and 28.8±18.8% of clot components, respectively, and 34.4±19.5% were red blood cells. In particular, clots were analyzed by a central lab blinded to clinical data. For patients treated with device 200 as a first-line device versus a second-line device, the extracted clots had lower average levels of red blood cells (29.04±14.56 for first-line vs 35.23±23.94 for second line) and high average levels of fibrin (35.26±10.80 for first-line vs 33.59±18.56 for second line). A substantial increase in collagen concentration is also present in clots retrieved from patients treated with device 200 as the first-line device compared to as a second-line device (2.37±3.94 for first-line vs 0.20±0.35 for second line). Although not wishing to be bound by theory, this change in clot composition may be attributed to the device 200 structure capable of pinching the tougher clots more efficiently when used as a first-line device compared to a comparative device being used as the first-line device.

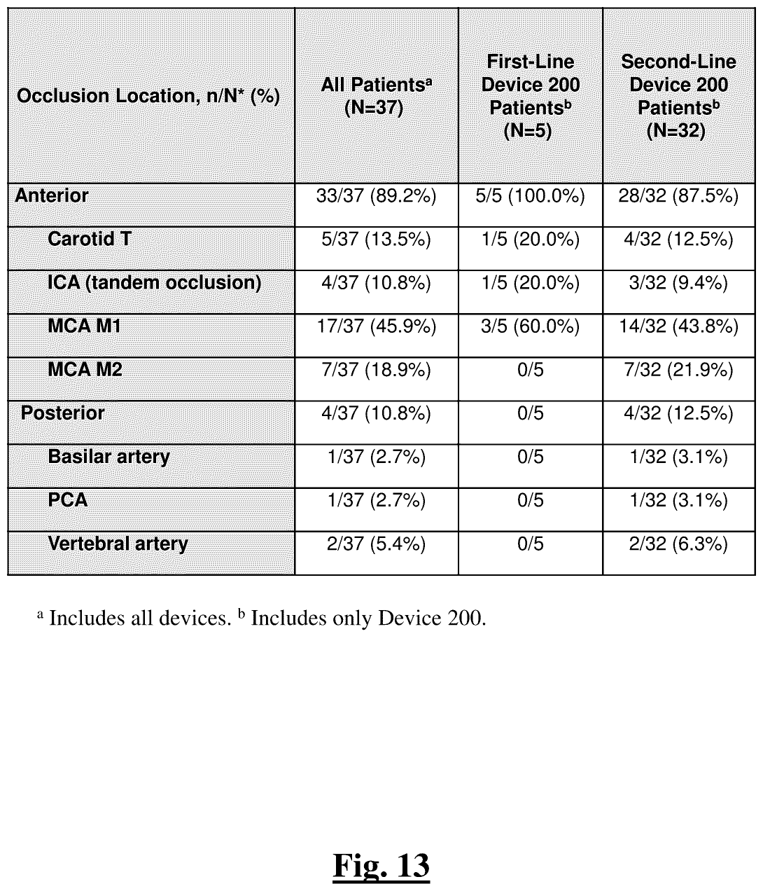

FIG. 13 provides clot locations for all patients of the study. The majority of patients of the study (89.2%; 33/37 patients) presented with anterior occlusive thrombus. Within the anterior locations, 45.9% (17/37) of all patients had occlusions located in the M1 segment of the middle cerebral artery (MCA) and 18.9% (7/37) had occlusions located in the M2 segment of the MCA. 5 out of 37 (13.5%) of patients had occlusions located at the carotid T and 4 out of 37 (10.8%) had tandem occlusions located at the internal carotid artery (ICA). The remaining patients (10.8%; 4/37) presented with posterior occlusive thrombus including basilar artery (1/37; 2.7%), posterior cerebral artery (PCA) (1/37; 2/7%), and vertebral artery (2/37; 5.4%). When device 200 was used as first line device, all clots retrieved were located in an anterior location, including carotid T, ICA, and MCA M1.

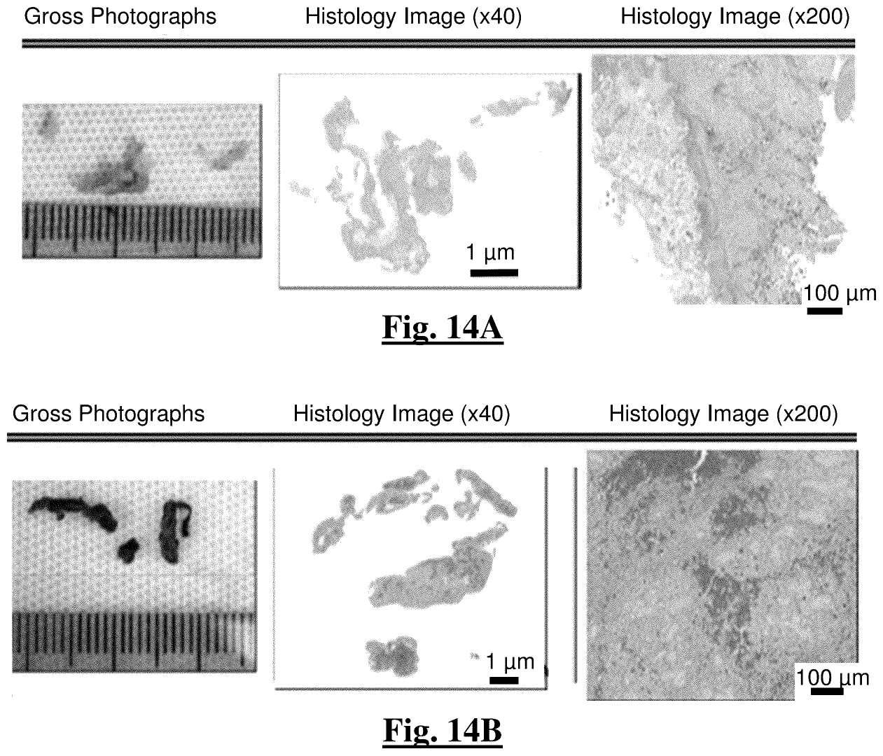

FIGS. 14A and 14B provide images and histology comparison of a typical clot and a tough clot retrieved in this study. The tough clot in FIG. 14A presented lower levels of red blood cells and higher levels of fibrin when compared to the typical clot (FIG. 14B). The retrieved clot of FIG. 14B had approximately a 1:1:1 distribution of red blood cells to fibrin to platelets/other. Whereas the retrieved clot of FIG. 14A had approximately 0.5 to 0.99:0.99-1.5:1 distribution of red blood cells to fibrin to platelets/other.

The clot retrieved in this study was richer in fibrin and platelet/other and poorer in red blood cells than levels typically reported in the literature. Similar composition profiles were observed in the overall cohort of clots which included fragments removed by all devices used in each case and the clot fragments removed specifically using device 200 as first-line or second-line treatment.

The extracted clot area and weight were similar in the overall and first-line device 200 cohorts. There was a trend to smaller clot in device 200 as second line and clots extracted with later passes were smaller.

The results of this series of patients with mainly fibrin- and platelet-rich clots support the utility of device 200 in cases that are typically challenging using standard approaches. Device 200 may be considered as a first-line device in patients with suspected or known tough clots.

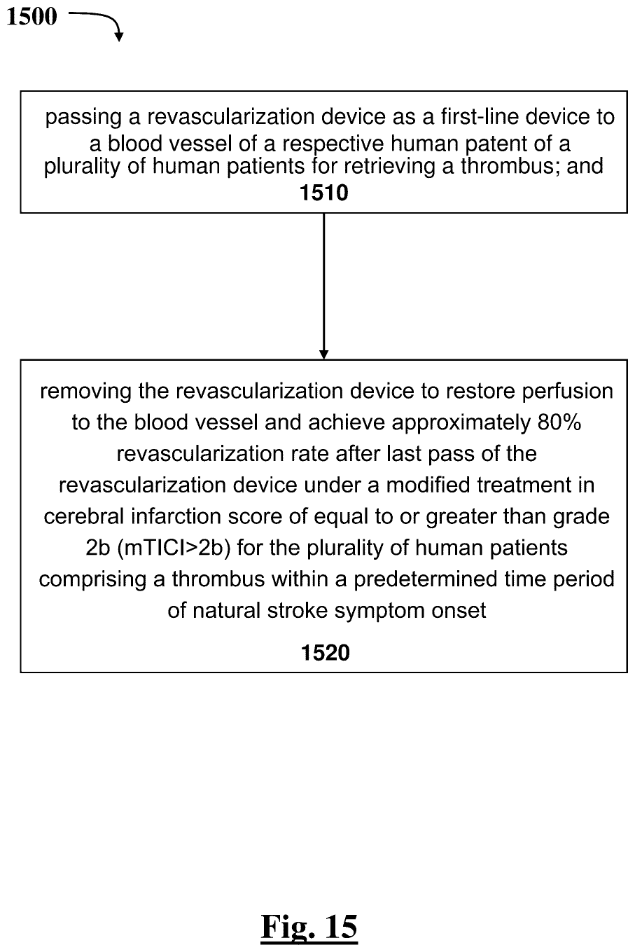

FIG. 15 depicts a method or use 1500 for treating ischemic stroke. The method 1500 can include delivering 1510 a revascularization device as a first-line device to a blood vessel of a respective human patent of a plurality of human patients for retrieving a thrombus; and restoring 1520 perfusion to the blood vessel by passing the revascularization device by, through, or about the thrombus and removing the revascularization device to achieve approximately 80% revascularization rate after last pass of the revascularization device under a modified treatment in cerebral infarction score of equal to or greater than grade 2b (mTICI≥2b) for the plurality of human patients with one or more cerebral occlusions within a predetermined time period of natural stroke symptom onset. Method 1500 can end after step 1520. In other embodiments, additional steps according to the examples described above can be performed.

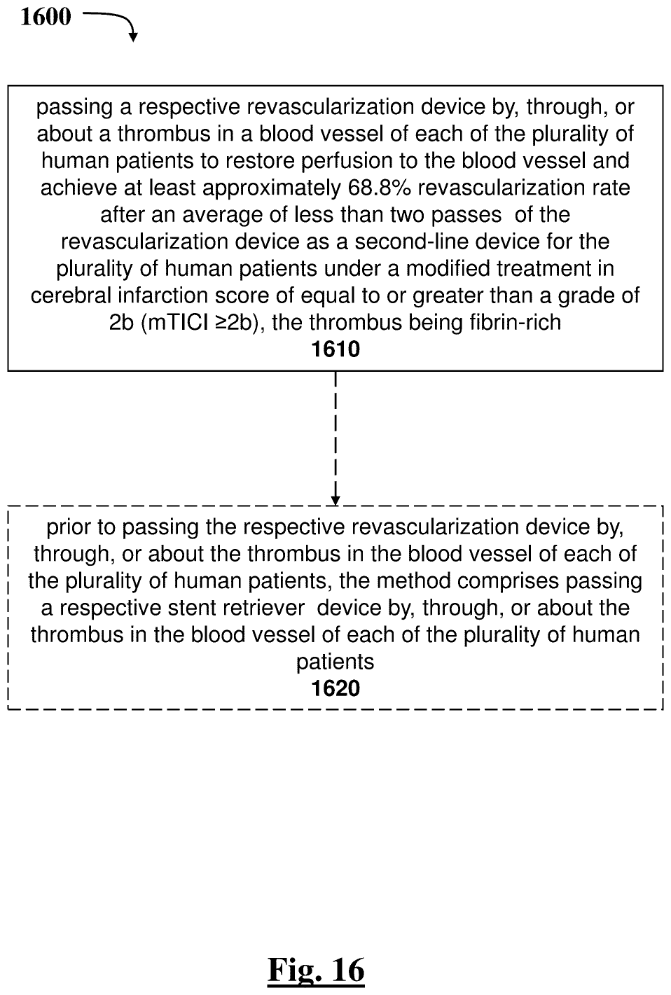

FIG. 16 depicts a method or use 1600 for treating ischemic stroke. The method 16Error! Reference source not found.00 can include passing 1610 a respective revascularization device by, through, or about a thrombus in a blood vessel of each of the plurality of human patients to restore perfusion to the blood vessel and achieve at least approximately 68.8% revascularization rate after an average of less than two passes of the revascularization device as a second-line device for the plurality of human patients under a modified treatment in cerebral infarction score of equal to or greater than a grade of 2b (mTICI≥2b), the thrombus being fibrin-rich. Optionally, method 1600 can include passing 1620 a respective stent retriever device by, through, or about the thrombus in the blood vessel of each of the plurality of human patients prior to passing the respective revascularization device by, through, or about the thrombus in the blood vessel of each of the plurality of human patients. Method 1600 can end after step 1610. In other embodiments, additional steps according to the examples described above can be performed.

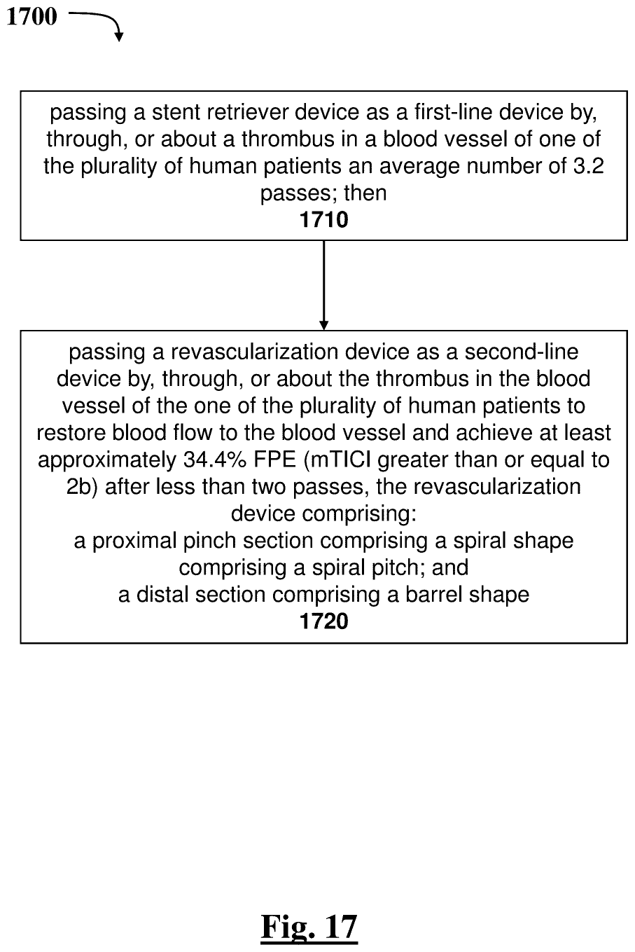

FIG. 17 depicts a method or use 1700 for treating ischemic stroke. The method 17Error! Reference source not found.00 can include passing 1710 a stent retriever device as a first-line device by, through, or about a thrombus in a blood vessel of one of the plurality of human patients an average number of 3.2 passes. Method 1700 can also include passing 1720 a revascularization device as a second-line device by, through, or about the thrombus in the blood vessel of the one of the plurality of human patients to restore blood flow to the blood vessel and achieve at least approximately 34.4% FPE (mTICI greater than or equal to 2b) after less than two passes. The revascularization device can include a proximal pinch section having a spiral shape comprising a spiral pitch, and a distal section comprising a barrel shape. Method 1700 can end after step 1720. In other embodiments, additional steps according to the examples described above can be performed.

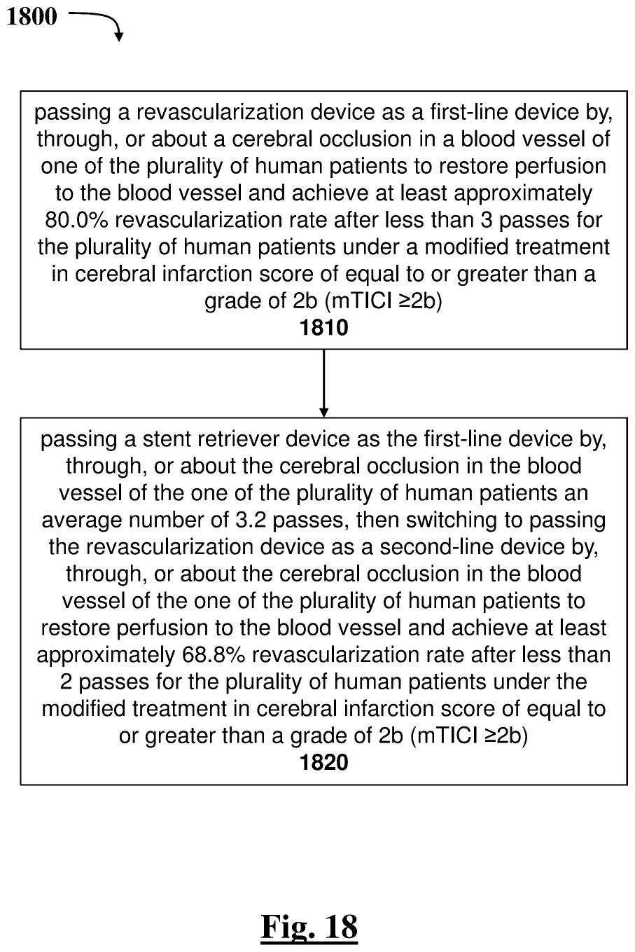

FIG. 18 depicts a method or use 1800 for treating ischemic stroke. The method 18Error! Reference source not found.00 can include passing 1810 a revascularization device as a first-line device by, through, or about a cerebral occlusion in a blood vessel of one of the plurality of human patients to restore perfusion to the blood vessel and achieve at least approximately 80.0% revascularization rate after less than 3 passes for the plurality of human patients under a modified treatment in cerebral infarction score of equal to or greater than a grade of 2b (mTICI≥2b). Method 1800 can also include passing 1820 a stent retriever device as the first-line device by, through, or about the cerebral occlusion in the blood vessel of the one of the plurality of human patients an average number of 3.2 passes, then switching to passing the revascularization device as a second-line device by, through, or about the cerebral occlusion in the blood vessel of the one of the plurality of human patients to restore perfusion to the blood vessel and achieve at least approximately 68.8% revascularization rate after less than 2 passes for the plurality of human patients under the modified treatment in cerebral infarction score of equal to or greater than a grade of 2b (mTICI≥2b). Method 1800 can end after step 1820. In other embodiments, additional steps according to the examples described above can be performed.

The device 200 and related methods of use of this disclosure demonstrated high rates of substantial reperfusion and functional independence in patients with acute ischemic stroke secondary to large-vessel occlusions. The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:
Clause 1. A method of restoring blood flow in neurovasculature of a human patient by removing thrombus in a plurality of human patients experiencing ischemic stroke, the method comprising: delivering a revascularization device as a first-line device to a blood vessel of a respective human patent of a plurality of human patients for retrieving a thrombus; and restoring perfusion to the blood vessel by passing the revascularization device by, through, or about the thrombus and removing the revascularization device to achieve approximately 80% revascularization rate after last pass of the revascularization device under a modified treatment in cerebral infarction score of equal to or greater than grade 2b (mTICI≥2b) for the plurality of human patients with one or more cerebral occlusions within a predetermined time period of natural stroke symptom onset.
Clause 2. The method of clause 1, further comprising detecting, prior to the step of passing the revascularization device as the first-line device, an initial revascularization rate of the respective human patent of the plurality of human patients less than a grade 2b (mTICI<2b).
Clause 3. The method of clause 1, wherein achieving approximately 80% revascularization rate for the plurality of human patients under the modified treatment in cerebral infarction score of equal to or greater than grade 2b (mTICI≥2b) comprises less than three passes of the revascularization device as the first-line device.
Clause 4. The method of clause 1, wherein achieving approximately 80% revascularization rate for the plurality of human patients under the modified treatment in cerebral infarction score of equal to or greater than grade 2b (mTICI≥2b) comprises an average of approximately 2.4 passes of the revascularization device as the first-line device.
Clause 5. The method of clause 1, further comprising passing the revascularization device by, through, or about the thrombus up to four passes prior to switching to a stent retriever device as a second-line device.
Clause 6. The method of clause 1, further comprising reducing, by the step of passing the revascularization device as the first-line device, post-procedural clinical complications selected from at least one of vessel dissection, vessel perforation, hematoma, emboli in new territory, infarction in new territory, or hemorrhagic transformation.
Clause 7. The method of clause 1, the method being performed within approximately 8 hours of stroke symptom onset.
Clause 8. The method of clause 1, the method being performed within approximately 11 hours of stroke symptom onset.
Clause 9. The method of clause 1, the cerebral occlusion being positioned in an internal carotid artery, a M1 segment and/or a M2 segment of a middle cerebral artery, a vertebral artery, or a basilar artery of the human patient.
Clause 10. The method of clause 1, the revascularization device having a collapsed delivery configuration and an expanded deployed configuration, the revascularization device comprising a proximal pinch section comprising a spiral shape comprising a spiral pitch; and a distal section comprising a barrel shape.
Clause 11. The method of clause 10, the revascularization device configured to position the thrombus against a wall of the vessel and then pinch the thrombus with the proximal pinch section.
Clause 12. The method of clause 10,

Clause wherein, in the expanded deployed configuration, the revascularization device comprises peaks of the proximal pinch section are laterally spaced-apart and when under tension, pinching the thrombus between the peaks.
Clause 13. The method of clause 10, the proximal pinch section comprising a plurality of cells defined by struts and crowns connected to corresponding struts and/or crowns, and wherein at least some of the struts and/or crowns of the proximal pinch section are aligned with a wave-like form to enhance embedding of clot.
Clause 14. The method of clause 10, the proximal pinch section comprising one or more clot gripping features.
Clause 15. The method of clause 10, the proximal pinch section is substantially curvilinear in the collapsed and expanded configurations.
Clause 16. The method of clause 10, the proximal pinch section comprising a transverse cross section between peaks comprising both flat and curved sections.
Clause 17. The method of clause 10, the proximal pinch section comprises a flat shape in transverse cross section.
Clause 18. A method of restoring blood flow in neurovasculature by removing thrombus in a plurality of human patients experiencing ischemic stroke, the method comprising: passing a respective revascularization device by, through, or about a thrombus in a blood vessel of each of the plurality of human patients to restore perfusion to the blood vessel and achieve at least approximately 68.8% revascularization rate after an average of less than two passes of the revascularization device as a second-line device for the plurality of human patients under a modified treatment in cerebral infarction score of equal to or greater than a grade of 2b (mTICI≥2b), the thrombus being fibrin-rich.
Clause 19. The method of clause 18, wherein prior to passing the respective revascularization device by, through, or about the thrombus in the blood vessel of each of the plurality of human patients, the method comprises passing a respective stent retriever device by, through, or about the thrombus in the blood vessel of each of the plurality of human patients.
Clause 20. The method of clause 19, further comprising: wherein after the step of passing the respective stent retriever device by, through, or about the thrombus in the blood vessel of each of the plurality of human patients, and wherein prior to passing the respective revascularization device by, through, or about the thrombus in the blood vessel of each of the plurality of human patients, the method comprises detecting a revascularization rate of the respective human patent of the plurality of human patients less than a grade 2b (mTICI<2b).
Clause 21. The method or use of Clause 20, further comprising switching to the revascularization device as the second-line device to restore perfusion to the blood vessel by passing the revascularization device by, through, or about the thrombus to achieve at least approximately 68.8% revascularization rate after an average of approximately 1.7 passes of the revascularization device for the plurality of human patients under the modified treatment in cerebral infarction score of equal to or greater than a grade of 2b (mTICI≥2b).
Clause 22. The method of clause 20, the revascularization device having a collapsed delivery configuration and an expanded deployed configuration, the revascularization device comprising a proximal pinch section comprising a spiral shape comprising a spiral pitch; and a distal section comprising a barrel shape.
Clause 23. The method of clause 22, the revascularization device configured to position the thrombus against a wall of the vessel and then pinch the thrombus with the proximal pinch section.
Clause 24. The method of clause 21, the step of passing the revascularization device comprises retracting the revascularization device, after being passed by, through or about the thrombus, while pinching the thrombus.
Clause 25. The method of clause 21, the revascularization device being configured to remove the thrombus or portions thereof that remain after passing the stent retriever device as a first-line device.
Clause 26. The method of clause 25, wherein the thrombus or portioned thereof are fibrin-rich.
Clause 27. The method of clause 21, the method being performed within approximately 8 hours of stroke symptom onset.
Clause 28. The method of clause 21, the method being performed within approximately 11 hours of stroke symptom onset.
Clause 29. A method of restoring blood flow in neurovasculature by removing thrombus in a plurality of human patients experiencing ischemic stroke, the method comprising: passing a stent retriever device as a first-line device by, through, or about a thrombus in a blood vessel of one of the plurality of human patients an average number of 3.2 passes; then passing a revascularization device as a second-line device by, through, or about the thrombus in the blood vessel of the one of the plurality of human patients to restore blood flow to the blood vessel and achieve at least approximately 34.4% FPE (mTICI greater than or equal to 2b) after less than two passes, the revascularization device comprising: a proximal pinch section comprising a spiral shape comprising a spiral pitch; and a distal section comprising a barrel shape.
Clause 30. The method of clause 29, the revascularization device further comprising a collapsed delivery configuration and an expanded deployed configuration, wherein, in the expanded deployed configuration, the revascularization device comprises peaks of the proximal pinch section are laterally spaced-apart and when under tension, the method comprising: pinching the thrombus between the peaks.
Clause 31. The method of clause 29, the proximal pinch section comprising a plurality of cells defined by struts and crowns connected to corresponding struts and/or crowns, and wherein at least some of the struts and/or crowns of the proximal pinch section are aligned with a wave-like form to enhance embedding of the thrombus.
Clause 32. The method of clause 29, the proximal pinch section comprising one or more thrombus gripping features.
Clause 33. The method of clause 29, the proximal pinch section is substantially curvilinear in the collapsed and expanded configurations.
Clause 34. The method of clause 29, the proximal pinch section comprises a transverse cross section between peaks comprising both flat and curved sections.
Clause 35. The method of clause 29, the proximal pinch section comprises a flat shape in transverse cross section.
Clause 36. The method of clause 29, the revascularization device being configured to remove the thrombus or portions thereof that are fibrin-rich.
Clause 37. The method of clause 29, the thrombus being located in at least one of the following locations in the human patient: a carotid artery, a M1 middle cerebral artery, a M2 middle cerebral artery, a basilar artery, and a vertebral artery.
Clause 38. The method of clause 29, the revascularization device configured for use within approximately 6 hours of stroke symptom onset.
Clause 39. The method of clause 29, the revascularization device configured for use within approximately 8 hours of stroke symptom onset.
Clause 40. The method of clause 29, the revascularization device configured for use within approximately 11 hours of stroke symptom onset.
Clause 41. A method of restoring blood flow in neurovasculature of a human patient by removing a cerebral occlusion in a plurality of human patients experiencing ischemic stroke, the method comprising at least one of: passing a revascularization device as a first-line device by, through, or about a cerebral occlusion in a blood vessel of one of the plurality of human patients to restore perfusion to the blood vessel and achieve at least approximately 80.0% revascularization rate after less than 3 passes for the plurality of human patients under a modified treatment in cerebral infarction score of equal to or greater than a grade of 2b (mTICI≥2b); or passing a stent retriever device as the first-line device by, through, or about the cerebral occlusion in the blood vessel of the one of the plurality of human patients an average number of 3.2 passes, then switching to passing the revascularization device as a second-line device by, through, or about the cerebral occlusion in the blood vessel of the one of the plurality of human patients to restore perfusion to the blood vessel and achieve at least approximately 68.8% revascularization rate after less than 2 passes for the plurality of human patients under the modified treatment in cerebral infarction score of equal to or greater than a grade of 2b (mTICI≥2b).
Clause 42. The method of clause 41, further comprising: detecting angiographically, after the step of passing the stent retriever device an average number of 3.2 passes as the first-line device, the revascularization rate for the plurality of human patients is less than grade 2b (mTICI<2b).
Clause 43. The method of clause 41, the step of passing the revascularization device as the first-line device or the second-line device comprises retracting the revascularization device, after being passed by, through or about the cerebral occlusion, while pinching the cerebral occlusion.
Clause 44. The method of clause 41, the revascularization device being configured to remove the cerebral occlusion or portions thereof that are fibrin-rich.
Clause 45. The method of clause 41, further comprising reducing, by the step of passing the revascularization device as the first-line device, post-procedural clinical complications selected from at least one of vessel dissection, vessel perforation, hematoma, emboli in new territory, infarction in new territory, or hemorrhagic transformation.
Clause 46. The method of clause 41, the method being performed within approximately 6 hours of stroke symptom onset.
Clause 47. The method of clause 41, the method being performed within approximately 8 hours of stroke symptom onset.
Clause 48. The method of clause 41, the method being performed within approximately 11 hours of stroke symptom onset.
Clause 49. The method of clause 41, the cerebral occlusion being positioned in an internal carotid artery, a M1 segment and/or a M2 segment of a middle cerebral artery, a vertebral artery, or a basilar artery of the human patient.
Clause 50. The method of clause 41, the revascularization device comprising a collapsed delivery configuration and an expanded deployed configuration, the revascularization device comprising a proximal pinch section comprising a spiral shape comprising a spiral pitch; and a distal section comprising a barrel shape, the revascularization device configured to pin the cerebral occlusion against a wall of the blood vessel and then pinch the cerebral occlusion with the proximal pinch section.

What is claimed is:

1. A method of restoring blood flow in neurovasculature of a human patient experiencing ischemic stroke, the method comprising:
identifying a respective human patient within a plurality of human patients at risk of comprising a thrombus;
passing a revascularization device as a first-line device to a blood vessel of the respective human patient of the plurality of human patients for retrieving a thrombus,
wherein the revascularization device comprises:
a collapsed delivery configuration and an expanded deployed configuration,
a proximal pinch section having a spiral shape comprising a spiral pitch and
a distal section comprising a barrel shape;
removing the revascularization device to restore perfusion to the blood vessel and achieve approximately 80% revascularization rate after last pass of the revascularization device under a modified treatment in cerebral infarction score of equal to or greater than grade 2b (mTICI>2b) for the plurality of human patients comprising a thrombus within a predetermined time period of natural stroke symptom onset.

2. The method of claim 1, further comprising detecting, prior to the step of passing the revascularization device as the first-line device, an initial revascularization rate of the respective human patient of the plurality of human patients less than a grade 2b (mTICI<2b).

3. The method of claim 1, wherein removing the revascularization device to restore perfusion to the blood vessel and achieve approximately 80% revascularization rate for the plurality of human patients under the modified treatment in cerebral infarction score of equal to or greater than grade 2b (mTICI≥2b) comprises less than three passes of the revascularization device as the first-line device.

4. The method of claim 1, wherein removing the revascularization device to restore perfusion to the blood vessel and achieve approximately 80% revascularization rate for the plurality of human patients under the modified treatment in cerebral infarction score of equal to or greater than grade 2b (mTICI≥2b) comprises an average of approximately 2.4 passes of the revascularization device as the first-line device.

5. The method of claim 1, further comprising reducing, by the step of passing the revascularization device as the first-line device, post-procedural clinical complications selected from at least one of vessel dissection, vessel perforation, hematoma, emboli in new territory, infarction in new territory, or hemorrhagic transformation.

6. The method of claim 1, the method being performed within at least 11 hours of stroke symptom onset.

7. The method of claim 1, the thrombus being positioned in an internal carotid artery, a M1 segment and/or a M2 segment of a middle cerebral artery, a vertebral artery, or a basilar artery of the human patient.

8. The method of claim 1, wherein prior to passing the revascularization device by, through, or about the thrombus in the blood vessel of each of the plurality of human patients, the method comprises passing a stent retriever device by, through, or about the thrombus in the blood vessel of each of the plurality of human patients.

9. The method of claim 1, the revascularization device configured to position the thrombus against a wall of the blood vessel and then pinch at least a portion of the thrombus with the proximal pinch section.

10. The method of claim 1 wherein, in the expanded deployed configuration, the revascularization device comprises peaks of the proximal pinch section are laterally spaced-apart and when under tension, pinching the thrombus between the peaks.

11. The method of claim 1, the proximal pinch section comprising a plurality of cells defined by struts and crowns connected to corresponding struts and crowns, and wherein at least some of the struts or the crowns of the proximal pinch section are aligned with a wave-like form to enhance embedding of clot.

12. The method of claim 1, the proximal pinch section comprises one or more clot gripping features.

13. A method of restoring blood flow in neurovasculature by removing thrombus in a plurality of human patients experiencing ischemic stroke, the method comprising:
passing a revascularization device by, through, or about a thrombus in a blood vessel of each of the plurality of human patients to restore perfusion to the blood vessel and achieve at least approximately 68.8% revascularization rate after an average of less than two passes of the revascularization device as a second-line device for the plurality of human patients under a modified treatment in cerebral infarction score of equal to or greater than a grade of 2b (mTICI≥2b), the thrombus being fibrin-rich.

14. The method of claim 13, wherein prior to passing the revascularization device by, through, or about the thrombus in the blood vessel of each of the plurality of human patients, the method comprises passing a stent retriever device by, through, or about the thrombus in the blood vessel of each of the plurality of human patients.

15. The method of claim 14, further comprising:
wherein after the step of passing the stent retriever device by, through, or about the thrombus in the blood vessel of each of the plurality of human patients, and wherein prior to passing the revascularization device by, through, or about the thrombus in the blood vessel of each of the plurality of human patients, the method comprises detecting a revascularization rate of a respective human patient of the plurality of human patients less than a grade 2b (mTICI<2b).

16. The method of claim 15, wherein prior to passing the revascularization device by, through, or about the thrombus in the blood vessel of each of the plurality of human patients, the method further comprises:
switching to the revascularization device as the second-line device to restore perfusion to the blood vessel by passing the revascularization device by, through, or about the thrombus to achieve at least approximately 68.8% revascularization rate after an average of approximately 1.7 passes of the revascularization device for the plurality of human patients under the modified treatment in cerebral infarction score of equal to or greater than a grade of 2b (mTICI≥2b).

17. The method of claim 13, the revascularization device comprising:
a collapsed delivery configuration and an expanded deployed configuration;
a proximal pinch section comprising a spiral shape comprising a spiral pitch; and
a distal section comprising a barrel shape,
wherein the revascularization device is configured to position the thrombus against a wall of the blood vessel and then pinch at least a portion of the thrombus with the proximal pinch section.

18. The method of claim 13, wherein the step of passing the revascularization device comprises retracting the revascularization device, after being passed by, through or about the thrombus, while pinching the thrombus.

19. The method of claim 14, wherein the revascularization device is configured to remove the thrombus or portions thereof that remain after passing the stent retriever device as a first-line device.

20. The method of claim 13, wherein the thrombus or portion thereof comprises more fibrin than red blood cells.
